(19) United States
(12) Patent Application Publication
(10) Pub. No.: US 2026/0026926 A1
(43) Pub. Date: Jan. 29, 2026

(54) ENDOSCOPIC PRINTHEAD FOR BIOPRINTING

(71) Applicant: THE ROYAL INSTITUTION FOR THE ADVANCEMENT OF LEARNING/MCGILL UNIVERSITY, Montréal (CA)

(72) Inventors: Swen GROEN, Montreal (CA); Luc MONGEAU, Mont Royal (CA); Audrey SEDAL, Montreal (CA); Jianyu LI, Verdun (CA)

(21) Appl. No.: 19/279,583

(22) Filed: Jul. 24, 2025

Related U.S. Application Data
(60) Provisional application No. 63/675,593, filed on Jul. 25, 2024.

(51) Int. Cl. A61F 2/04; A61B 1/015; A61B 1/267; B33Y 30/00

(52) U.S. Cl. CPC A61F 2/04; A61B 1/015; B33Y 30/00; A61B 1/267; A61F 2002/046; A61F 2240/002

(57) ABSTRACT

An endoscopic printing device comprising: a printing head having: a nozzle for outputting a biomaterial; a central conduit connectable to a source of the biomaterial and connected to the nozzle, the central conduit being flexible; ribs disposed around the central conduit and spaced apart from one, a rib of the ribs defining three or more guiding apertures extending axially through the rib, the ribs being interconnected to one another; and cables extending through the guiding apertures, ends of the cables attached to the central conduit, the cables configured to be engaged to an actuator for controlling motion of the endoscopic printing head; and an actuation system engaged to the cables, the actuation system including actuators each engaged to a respective one of the cables, the actuators operable to vary a tension in one or more of the cables relative to a remainder of the cable to bend the central conduit.

### CROSS-REFERENCE TO RELATED APPLICATIONS

This application claims priority from U.S. patent application 63/675,593 filed on Jul. 25, 2024, the entire contents of which are incorporated by reference herein.

### GOVERNMENT SUPPORT

This invention was made with government support under grant 5R01DC018577 awarded by the National Institutes of Health. The government has certain rights in the invention.

### TECHNICAL FIELD

The application relates generally to medical devices and, more particularly, to endoscopic devices for bioprinting material in-situ.

### BACKGROUND

Bioprinting is a fabrication method involving layered or volumetric deposition of biomaterials to create complex structures. It may be used for tissue engineering, regenerative medicine, and drug screening applications. Bio fabrication may reduce the complexity and the cost of fabrication of soft tissue implants. Due to multi-material construction, and incorporation of growth factors, complex constructs mimicking the native tissue can be created. The created constructs can be used as implants to mimic native tissue and enhance tissue regeneration in situ. Printing these constructs ex situ is challenging in a clinical setting. Construct handling may result in contamination, increasing the risk of infections, and inflammation. Moreover, the necessary handling may result in the deformation of the structure, creating a geometric mismatch between the structure and target tissue. Furthermore, the resolution of scanning methods before surgery, tissue movement, and surgical interactions may result in a deviation between the scanned defect and the actual defect. Due to the difference between the actual defect and printed construct, chances increase for a geometric mismatch, which leads to challenges during the implantation process. Therefore, improvements are sought.

### SUMMARY

Three-dimensional printing may be used for construct fabrication in tissue engineering and regenerative medicine. Current in vitro bioprinting approaches may result in geometric mismatch and contamination. In situ bioprinting may reduce these drawbacks by creating the constructs at the target tissue, using handheld and robotic printers. Current approaches for in situ bioprinting require open surgery. A novel approach to use minimally invasive surgery is the use of continuum robot designs for in situ bioprinting. The present disclosure introduces an endoscopic printhead for in situ bioprinting using cable-driven actuation.

The proposed design of an endoscopic printhead may reduce invasiveness for in situ bioprinting. The results indicate a low error for the kinematic model and show the ability to print different hydrogels. The disclosed device may also increase the feasibility in a clinical setting due to lower chances of contamination and complications.

In one aspect, there is provided an endoscopic printing head for bioprinting, comprising: a nozzle for outputting a biomaterial to be printed; a central conduit extending along a longitudinal axis and fluidly connectable to a source of the material, the central conduit fluidly connected to the nozzle, the central conduit being flexible; ribs mounted to the central conduit and spaced apart from one another along the longitudinal axis, the ribs including a rib defining three or more guiding apertures extending axially through the rib and being circumferentially distributed around the longitudinal axis; an outer shell extending around the ribs; and cables extending through the guiding apertures in the ribs, ends of the cables attached to the central conduit proximate the nozzle, the cables configured to be engaged to an actuator for controlling motion of the endoscopic printing head.

The endoscopic printing head described above may include any of the following features, in any combinations.

In some embodiments, the outer shell are made of a resin polymer material.

In some embodiments, the central conduit and the outer shell are made of an elastic material.

In some embodiments, the elastic material is silicon.

In some embodiments, spaces defined between the outer shell, the central conduit, and the ribs are filled with the elastic material.

In some embodiments, the ends of the cables are secured to the nozzle, the ends of the cables secured to the central conduit via the nozzle.

In some embodiments, the rib defines a central aperture for receiving the central conduit.

In some embodiments, the central aperture communicates with the guiding apertures.

In some embodiments, the central aperture is separated from the guiding apertures.

In some embodiments, the central conduit defines annular grooves distributed along the longitudinal axis, each of the ribs received within a respective one of the grooves.

In another aspect, there is provided an endoscopic printing device comprising: a printing head as defined; and an actuation system engaged to the cables, the actuation system including actuators each engaged to a respective one of the cables, the actuators operable to vary a tension in one or more of the cables relative to a remainder of the cable to bend the central conduit.

In another aspect, there is provided a method of manufacturing an endoscopic printing head for bioprinting a biomaterial, comprising: obtaining a central conduit extending along a longitudinal axis; disposing ribs around the central conduit, the ribs distributed axially along the longitudinal axis, the ribs defining guiding apertures circumferentially distributed around the longitudinal axis; passing cables through the guiding apertures of the ribs and securing ends of the cables to the central conduit proximate an outlet thereof; and enclosing the central conduit and the ribs with an outer shell.

The method described above may include any of the following features, in any combinations.

In some embodiments, the obtaining of the central conduit includes obtaining the central conduit having a nozzle secured thereto at the outlet of the central conduit.

In some embodiments, the method includes securing a nozzle to the outlet of the central conduit.

In some embodiments, the securing of the ends of the cables to the central conduit includes securing the ends of the cables to the nozzle, the cables secured to the central conduit via the nozzle.

In some embodiments, the disposing of the ribs around the central conduit includes inserting the central conduit through central apertures defined by the ribs.

In some embodiments, the method includes disposing each of the ribs within a respective one of annular grooves distributed longitudinally on the central conduit along the longitudinal axis.

In some embodiments, the obtaining of the central conduit includes molding the central conduit.

In some embodiments, the molding of the central conduit includes: disposing a rod inside a first mold having a shape corresponding to that of the central conduit; injecting an elastic material in the mold and around the central conduit; and removing the rod to define a central passage of the central conduit.

In some embodiments, the disposing of ribs around the central conduit includes: obtaining a core extending along the longitudinal axis, the core defining a central passage and ribs located outwardly of the central passage; and inserting the central conduit through the central passage of the core.

In some embodiments, the enclosing of the central conduit and the ribs with the outer shell includes: disposing the central conduit and the ribs in a second mold after the passing of the cables through the guiding apertures; and injecting the elastic material into the second mold thereby creating the outer shell and filling spaces defined between the ribs.

In some embodiments, the method includes inserting tubes into the guiding apertures, the passing of the cables through the guiding apertures includes passing the cables into the tubes, the method further comprising removing the tube after the injecting of the elastic material into the second mold.

In yet another aspect, there is provided a method of fabricating a biologically-compatible construct onto a target tissue, comprising performing in-situ bioprinting using the endoscopic printing device described above, including displacing the nozzle of the endoscopic printing head in three-dimensional space to print the biologically-compatible construct on the target tissue.

In one aspect, there is provided an endoscopic printing device comprising: a printing head having: a nozzle for outputting a biomaterial to be printed; a central conduit extending along a longitudinal axis and fluidly connectable to a source of the biomaterial, the central conduit fluidly connected to the nozzle, the central conduit being flexible; ribs disposed around the central conduit and spaced apart from one another along the longitudinal axis, a rib of the ribs defining three or more guiding apertures extending axially through the rib and being circumferentially distributed around the longitudinal axis, the ribs being interconnected to one another; and cables extending through the guiding apertures, ends of the cables attached to the central conduit proximate the nozzle, the cables configured to be engaged to an actuator for controlling motion of the endoscopic printing head; and an actuation system engaged to the cables, the actuation system including actuators each engaged to a respective one of the cables, the actuators operable to vary a tension in one or more of the cables relative to a remainder of the cable to bend the central conduit.

The endoscopic printing device described above may include any of the following features, in any combinations.

In some embodiments, the ribs are interconnected to one another by longitudinal members extending axially relative to the longitudinal axis from one of the ribs to an adjacent one of the ribs.

In some embodiments, the longitudinal members include a pair of longitudinal members between a first rib and a second rib of the ribs, longitudinal members of the pair being circumferentially offset from one another by about 120 degrees, the pair of the longitudinal members being clocked relative to a second pair of the longitudinal members disposed between the second rib and a third rib.

In some embodiments, the ribs define central passages sized to receive the central conduit, the guiding apertures opening to the central passages.

In some embodiments, the central conduit is a tube received through the central passages of the ribs, the cables being isolated from one another by a cooperation of the ribs and the tube.

In some embodiments, the ribs are interconnected to one another by an outer shell extending around and secured to peripheries of the ribs.

In some embodiments, the outer shell is made of an elastic material.

In some embodiments, spaces are defined between the outer shell, the central conduit, and the ribs are filled with an elastic material.

In some embodiments, the central conduit is separated from the guiding apertures.

In some embodiments, the ends of the cables are secured to the nozzle, the ends of the cables secured to the central conduit via the nozzle.

In yet another aspect, there is provided an endoscopic printing head for bioprinting, comprising: a nozzle for outputting a biomaterial to be printed; a central conduit extending along a longitudinal axis and fluidly connectable to a source of the biomaterial, the central conduit fluidly connected to the nozzle, the central conduit being flexible; ribs disposed around the central conduit and spaced apart from one another along the longitudinal axis, a rib of the ribs defining three or more guiding apertures extending axially through the rib and being circumferentially distributed around the longitudinal axis, the ribs being interconnected to one another; cables extending through the guiding apertures, ends of the cables attached to the central conduit proximate the nozzle, the cables configured to be engaged to an actuator for controlling motion of the endoscopic printing head.

The endoscopic printing head described above may include any of the following features, in any combinations.

In some embodiments, the ribs are interconnected to one another by longitudinal members extending axially relative to the longitudinal axis from one of the ribs to an adjacent one of the ribs.

In some embodiments, the longitudinal members include a pair of longitudinal members between a first rib and a second rib of the ribs, longitudinal members of the pair being circumferentially offset from one another by about 120 degrees, the pair of the longitudinal members being clocked relative to a second pair of the longitudinal members disposed between the second rib and a third rib

In some embodiments, the ribs define central passages sized to receive the central conduit, the guiding apertures opening to the central passages.

In some embodiments, the central conduit is a tube received through the central passages of the ribs, the cables being isolated from one another by a cooperation of the ribs and the tube.

In some embodiments, the ribs are interconnected to one another by an outer shell extending around and secured to peripheries of the ribs.

In some embodiments, the outer shell is made of an elastic material.

In some embodiments, spaces defined between the outer shell, the central conduit, and the ribs are filled with an elastic material.

In some embodiments, the central conduit is separated from the guiding apertures.

### BRIEF DESCRIPTION OF THE DRAWINGS

Reference is now made to the accompanying figures in which:

FIG. 1A is a three dimensional view of an endoscopic printing device;

FIG. 1B is a three dimensional view of an exemplary setup of the endoscopic printing device used for performing vocal folds repair;

FIG. 1C is a three dimensional view of an endoscopic printing device of another embodiment;

FIG. 2 is a three dimensional view of a printing head for the printing device of FIG. 1A or 1C;

FIG. 3 is a three dimensional exploded view of the printing head of FIG. 2;

FIG. 4 is a cross-sectional view taken along line 4-4 of FIG. 3;

FIG. 5 is a flowchart illustrating steps of manufacture the printing head of FIG. 2;

FIGS. 6A to 6K are three dimensional views illustrating different steps of a manufacturing process of the printing head of FIG. 2;

FIG. 7 is a side view of a printing head in accordance with another embodiment;

FIG. 8 is a three dimensional view of the printing head of FIG. 7 illustrating a plurality of positions thereof;

FIG. 9 is a three dimensional view of a core of the printing head of FIG. 8;

FIG. 10 is a side view of the printing head of FIG. 7;

FIGS. 10A to 10C are cross-sectional views taken along lines 10A-10A, 10B-10B, and 10C-10C on FIG. 10;

FIG. 11 is a cross-sectional view taken along line 11-11 of FIG. 10; and

FIGS. 12A to 12F are three dimensional views illustrating different steps of a manufacturing process of the printing head of FIG. 7.

### DETAILED DESCRIPTION

### Introduction

In situ bioprinting is a process in which a wound graft or tissue filler is fabricated directly on the target tissue. This may reduce the chances of contamination, geometric mismatch, and may eliminate construct handling. In situ bioprinting may enable crosslinking between native tissue and bio-ink, which may improve tissue-construct adhesion and using the native environment as a bioreactor for tissue regeneration. Minimal invasiveness may reduce the risk of post-surgery complications, recovery times and risk of infection.

Current methods consist of handheld printing methods and robotic printing methods to address these requirements. Handheld printing is controlled by the surgeon directly, while depositing hydrogels, which offers real-time control, increased flexibility and reduced system complexity. Due to the direct control of the surgeon, such existing methods are skill-dependent with resulting reduced accuracy. Both handheld and robotic methods, all described methods require open-field surgery, increasing the risk of contamination and recovery times. A near infrared bioprinting non-invasive method was proposed, but is limited to superficial tissues due to penetration depth.

A possible usage of bioprinting is phonosurgery, a process via which removal of large lesions may require the local delivery of a wound filling biomaterial, such as a hydrogel. Accurate deposition of hydrogels in situ remains challenging, reducing therapeutic efficiency. In-situ bioprinting may provide efficient and accurate delivery for skin, bone and, muscle tissue. To perform such operations, a minimally invasive flexible endoscopic printhead may be used and is described below.

An example of such procedures is the removal of vocal fold pathologies such as polyps, lesions, and cancers. The efficacy of these treatments is reduced due to post-surgical fibrosis. Post surgical fibrosis (PSF) occurs for 5-18% of patients. Fibrosis increases the stiffness of the vocal folds, disrupting the mucosal wave required for phonation. The fibrosis and increased vocal fold stiffness leads to reduced voice quality, with cases reporting a decrease in voice quality after surgery. PSF leads to additional treatment to increase the voice quality, increasing the burden on both the patient and healthcare. Reducing the risk of PSF is expected to increase the voice quality for patients, resulting in increased quality of life and reduced burden on healthcare.

The prevention of PSF may include hydrogel injections into the lamina propria. The hydrogel injection provides a structural network mimicking the mechanical vocal fold properties. The hydrogel promotes healthy tissue regeneration and degrades over time, thereby reducing fibrosis. Present delivery methods offer challenges for successful injections. Currently, injections are performed percutaneously. Without precise control of hydrogel placement, injections can be less efficient, maintaining risk of PSF. The lack of precise control results in the inability to inject into the superficial lamina propria, limiting the treatment options for superficial lesions. The pliability of the superficial lamina propria is important for phonation. The ability to precisely distribute the hydrogel over diverse lesion shapes and sizes while reconstructing natural vocal fold geometry could lead to better patient outcomes.

In-situ bioprinting directly deposits hydrogels in the target tissue and may provide accurate position control enabling the reconstruction of the desired tissue geometry. Additionally, minimally invasive in-situ bioprinting can reduce post-surgical complications. Minimally invasive procedures use natural orifices or small incisions to decrease risk of contamination and complications.

The present disclosure focuses on a bioprinting head that may be used for performing in-situ bioprinting. The head may be compatible with micro laryngeal surgery and may reduce required training wile facilitating the use of existing infrastructure and workflows. The standard for micro laryngeal surgery is suspension laryngoscopy. A laryngoscope is inserted transorally, providing a 10.5-13.5 mm diameter working channel, and fixed in place. The fixed laryngoscope facilitates bimanual operation and direct magnified visualization on the vocal folds. The procedure has been shown to be efficient with minimal post-procedural discomfort.

Some bioprinting methods are not compatible with suspension laryngoscopy. The disclosed head may be compatible with suspension laryngoscopy. This disclosure presents and evaluates a minimally invasive in-situ bioprinter (MIISB) design for vocal fold repair. The MIISB was designed for accurate deposition of hydrogel on the vocal folds. The MIISB may contain a soft robot printhead having a diameter of about 2.7 mm, which controls the location of hydrogel deposition. The MIISB was designed to be compatible with suspension laryngoscopy. This may allow the surgeon to accurately control the position and distribution of hydrogel deposition in real-time. Additionally, visualization of the vocal folds is maintained.

Control of the MIISB is achieved through a data-driven kinematic model within a 20 mm workspace. The MIISB achieved an accuracy of 1.33 mm. Control of the location was achievable with high repeatability, <0.2 mm, and low position drift, <0.8 mm. Deposition of a hyaluronic acid hydrogel was possible with a resolution of 1.2 mm. A demonstration showed ability to create single-layer and multilayer constructs on a flat surface. A surgical model was used to show the ability to reconstruct the vocal fold geometry accurately. The results indicate the ability to accurately control the position and distribution of hydrogel deposition. The MIISB shows the ability to improve on the quality of hydrogel injections. Improved hydrogel deposition may lead to reduced PSF, improving the quality of life of patients.

A data-driven model for real-time control was developed. The model resulted in highly accurate control of the nozzle-position with a 1.33 mm position error. The actual printing resolution was measured to be 1.2 mm. Functionality of the design was demonstrated by printing 20 mm constructs on a flat surface, comparable to the vocal fold size. Printing functionality was further demonstrated by recreating the vocal fold geometry on simulated defects. The results indicate the feasibility for accurate control on the deposition location and distribution of hydrogels on vocal folds.

### Bioprinting Device

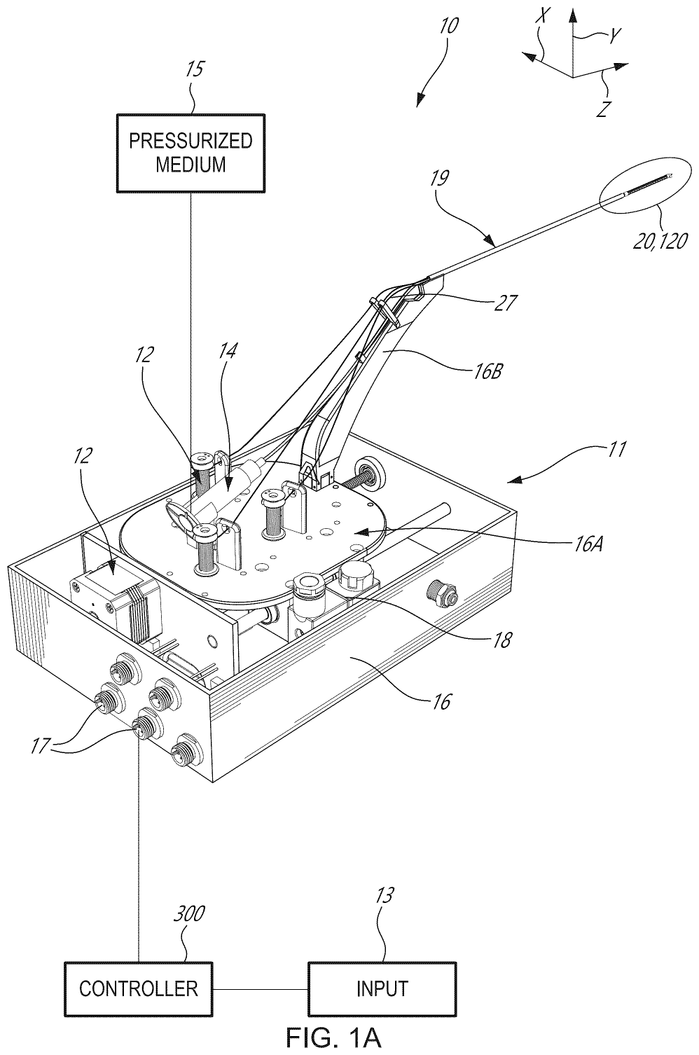

Referring to FIG. 1A, an endoscopic printing device is shown at 10. The device 10 includes an actuation system 11 operatively connected to a printing head 120. The actuation system 11 is configured to control motion of the printing head 120 as will be explained below. The actuation system 11 may include actuators 12 and an input device 13 configured for controlling the actuators 12. In the embodiment shown, the actuation system 11 includes a receptacle 14 filled with hydrogel or any suitable biomaterial to be printed using the printing head 120. The receptacle 14 may be a syringe or any suitable means configured for containing the hydrogel. Means are used to push the hydrogel towards the printing head 120. These means may include, for instance, a piston and a cylinder. An actuator may alternatively be used to push the hydrogel. A pressurized medium may be used for that purpose. In this embodiment, the receptacle 14 is fluidly connected to a pressurized medium 15, such as a reservoir of a pressurized gas, such as air. The pressurized medium 15 may be a pump in some embodiments. Any means operable to generate sufficient pressure to induce a flow of the hydrogel to the printing head 120 is contemplated without departing from the scope of the present disclosure.

The actuation system 11 includes a housing 16 onto which are mounted the actuators 12. Connectors 17 are secured to the housing 16 and are configured to operatively connect a controller 300 used to control operation of the printing device 10. The controller is configured to translate inputs provided by the input device 13 to the different actuators 12 to generate the desired movements of the printing head along directions X, Y, Z.

Five M12 connectors, although any suitable connectors may be used, may be used to connect the stepper motors and solenoid valve to their respective drivers. The housing 16 may include a platform 16A onto which are mounted the actuators 12, which may each include a stepper motor and a drum. Any suitable actuators may be used without departing from the scope of the present disclosure. Cables 27 responsible for actuation of the printing head 120 may be wound around those drums. Tension in the cable may be varied by powering the stepper motor. Actuation of the stepper motors and solenoid may be achieved through a wireless controller, PC and electronics housing. The electronics housing contains the drivers for the stepper motors and solenoid valve. The use of a separate electronics housing may allow for minimized intrusion on the surgeon's workspace. More detail about operation of the printing head 120 are presented below.

Referring to FIGS. 1A and 1B, a beam 16B protrudes from the platform 16A both vertically and horizontally away therefrom along directions Y and Z. As shown more distinctly in FIG. 1B, the beam 16B as a base at the platform and a tip that exceeds a footprint of the housing 16. In the context of vocal fold repairs, the device 10, including the beam 16B, may be positioned in such a way that a medical practitioner may use a microscope M, or other suitable visualisation tool, to operate the printing head 120 while seeing what is being printed on a patient P by the printing head 120. Understandably, a different setup may be used depending on the desired application and location where hydrogel application is required. To further increase the reach of the device, a tube 19 (e.g., stainless steel tube) protrudes from the tip of the beam 16B towards the patient P and the printing head 120 is secured to an extremity of the tube 19. The tube is in fluid communication with the receptacle 14 for flowing the hydrogel to the printing head 120. The tube 19 may have an outer diameter of about 3.2 mm and a length of about 250 mm, although other dimensions are contemplated depending of the intended use. The hollow stainless-steel tube houses the extrusion channel and cables and may enable bulk rigid body motion of the endoscopic printhead towards the target location. The total length of the stainless-steel tube and the endoscopic printhead may be a about 300 mm.

The beam 16B, which may be referred to as a strut, may make a 35° angle with the platform 16A. The angled strut may allow for the installation of the actuator housing under a surgical microscope M, thereby limiting possible intrusion within the surgeon's workspace. The strut was designed to be compatible with a Leica M525 F50 microscope, but may be designed to work with other apparatus. The stepper motors, the drums coiling the cables, cable guides, and the pressurized syringe are all mounted directly onto the linear stage and so move synchronously. The linear stage controls the axial endoscopic printhead position through a linear stepper motor. The linear stage is actuated to change the axial position, along direction Z, of the nozzle and is also used to compensate for axial displacement due to bending. With a linear actuator providing compensation, the nozzle tip can stay within a plane, normal to the axial centerline. Control of hydrogel is achieved through a solenoid valve connected to the syringe. The actuator housing can be mounted on a positioning arm, allowing the actuator housing to be moved and fixed in desired locations.

A valve 18, such as a solenoid valve or any other suitable valve, is fluidly connected to the pressurized medium 15. The valve 18 is operatively connected to the controller 300 and has open and closed configurations to selectively allow or prevent the pressurized medium from exerting a pressure force on the hydrogel. The valve may have a plurality of open positions each having a respective flow area different than one another to control a flow rate of the pressurized medium. This, in turn, may be used to vary a flow rate of the hydrogel being printed by the printing head 120. In the case of the pressurized medium being replaced by a pump, such a valve may be omitted and the flow rate may be controlled by the controller causing an actuation of the pump at a desired speed to obtain the desired flow rate of hydrogel.

Referring to FIG. 1B, the envisioned workflow to decrease PSF consists of two procedures. In the first procedure the source of voice abnormality is removed in accordance with current standards. This leads to acute inflammation and flow of blood. Once acute inflammation has subsided, a secondary procedure is required to reconstruct the vocal fold geometry. During the second procedure the patient will undergo general anesthesia. Afterwards, a laryngoscope L is fixed in place to visualize the vocal folds and create a working channel for the surgeon. The printhead is then directed to the vocal folds using the positioning arm. The surgeon fixes the positioning arm to prevent unintended movements during the operation. Using the controller the surgeon then directs the nozzle to the target location, maintaining vision through a surgical microscope. The surgeon then deposits the hydrogel to reconstruct the vocal fold geometry by controlling the nozzle position. Once finished, the surgeon moves the printhead to the neutral position for safe retraction. The printer is retracted using the positioning arm. The procedure is finished according to the standard procedures.

When operating on a submucosal lesion, a microflap procedure may be performed to access the injection site. A superficial incision is made prior to placement of the printer. The surgeon then continues the procedure as described above. When the defect is reconstructed with the hydrogel, the mucosal flap is coapted to finish the procedure. It will be appreciated that the devices disclosed herein are not limited to this specific application and may be used for other procedures, such as repairing other tissue such as heart tissue, stomach tissue, and so on.

Referring to FIG. 1C, another embodiment of a printing device is illustrated and shown at 110. Device 110 includes an actuation system 111 operatively connected to a controller 300. The actuation system 111 includes a housing 116 onto which are mounted actuators 112 as described above. In this embodiment, the device 110 includes an input device 113 mounted to the housing 116 and operable to control the actuators 112. A printing head 20 is disposed at a tip of a beam. As for the device 10 described above, the actuators 112 are operable to control movements of the printing head 20 relative to axes X, Y, Z. Device 110 may be equipped with the printing head 120 instead of the printing head 20. The same goes for the device 10. Both printing heads are described below.

### Bioprinting Head

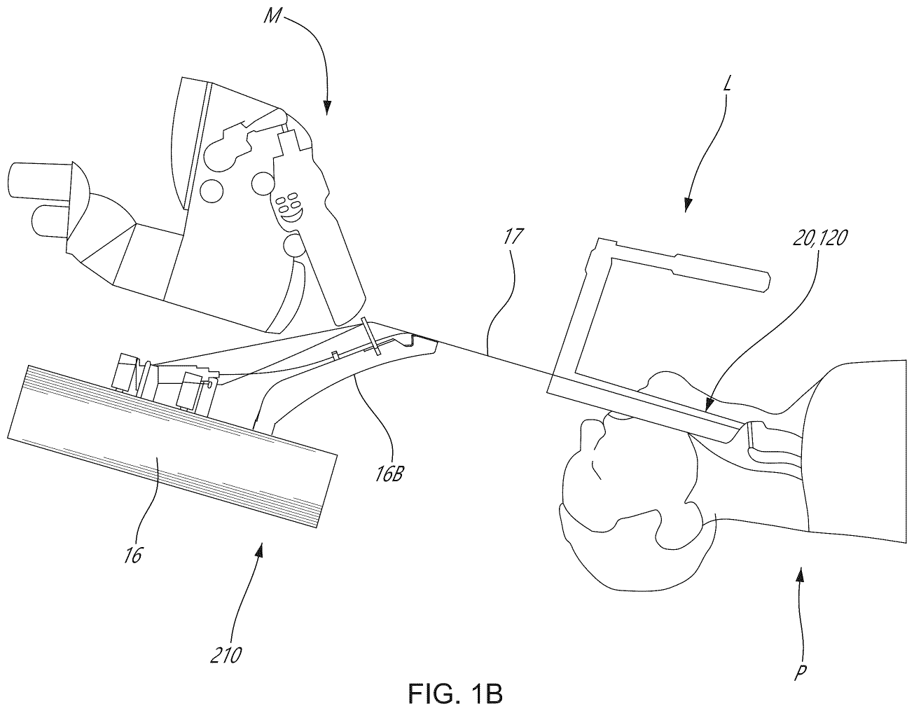

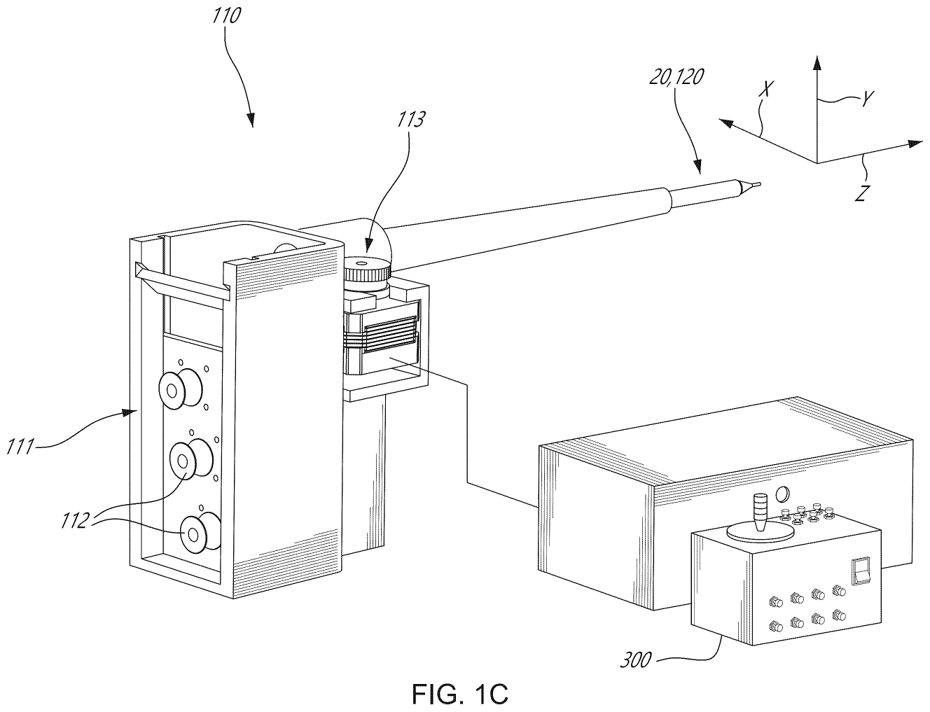

Referring to FIGS. 2-3, the printing head 20 is described in more detail. The printing head 20 has a nozzle 21 for outputting a material to be printed. The nozzle 21 may define a frusto-conical shape and includes at a tip 21A at an outlet of the nozzle 21. The tip 21A defines an opening for outputting the material to be printed. The nozzle 21 may be made of resin whereas the tip 21A may be a tube made of a metallic material such as stainless steel. The tip 21A may be an 18-gauge stainless tube of a length of about 8 mm. In the context of the present disclosure, the expression “about” implies variations of plus or minus 10%. The tip 21A may be embedded in the nozzle 21 using Loctite™, but any other fastening means may be used. In the embodiment shown, the nozzle 21 defines nozzle apertures 21B circumferentially distributed about the longitudinal axis L1. These nozzle apertures 21B may be used to route cables as will be discussed further below.

The printing head 20 further has a central conduit 22 extending along a longitudinal axis L1 and fluidly connectable to a source S (FIG. 1) of a fluid to be printed. The central conduit 22 is fluidly connected to the nozzle 21. The central conduit 22 is flexible. It may be made of any suitable material allowing flexibility such as, for instance, an elastic material such as a hyper-elastic material such as rubber, elastomer, silicon (e.g., Ecoflex 00-50™, Smooth on), and son on. In this embodiment, the central conduit 22 defines annular grooves 22A distributed along the longitudinal axis L1.

Referring more particularly to FIG. 3, the printing head 20 includes ribs 23 mounted to the central conduit 22 and distributed axially along the longitudinal axis L1. In this embodiment, each of the ribs 23 is received within a respective one of the annular grooves 22A of the central conduit 22. In this embodiment, the ribs 23 are equidistantly spaced apart from one another. This may not be the case in some embodiments. The ribs 23 may be resin disks (e.g., 8K standard resin). The ribs 23 may be about 1 mm think and may have a diameter of 7 mm. They may be spaced apart from one another by 5 mm. These dimensions may vary in some embodiments. The ribs 23 define each a central aperture 23A for receiving the central conduit 22.

In the embodiment shown, a tube 24 is inserted in the central conduit 22 from an opposite end of the tip 21A. The tube 24 may be made of stainless steel, but other suitable materials are contemplated. The tube 24 may have a length of about 20 mm and may be inserted a depth of about 5 mm into the central conduit 22. It may be secured using Loctite™, but other fastening means are contemplated. A feeding tube 25, which may be made of plastic and which may have an internal diameter of about 1.27 mm, is secured to the central conduit 22 proximate an inlet thereof via the tube 24. In other words, the feeding tube 25 abuts a proximal end of the central conduit 22, and both of a portion of the central conduit 22 and the feeding tube 25 each receive a respective portion of the feeding tube 25 therein. A tight fit may be provided between the feeding tube 25 and the tube 24, and between the tube 24 and the central conduit 22. Glue or other fastening means, such as a clamp, may be used in some embodiments.

An outer shell 26 is disposed around the ribs 23 and around the central conduit 22. The outer shell 26 may be made of the same material as the central conduit 22 to allow flexibility. This material may be, for instance, an elastic material, such as a hyper-elastic material such as rubber, elastomer, silicon, and son on. A different material may be used for the outer shell 26 than for the central conduit 22. In the embodiment shown, spaces defined between the outer shell 26, the central conduit 22, and the ribs 23 are filled with the same material as that of the outer shell 26, which may be an elastic material, such as a hyper-elastic material such as rubber, elastomer, silicon, and son on.

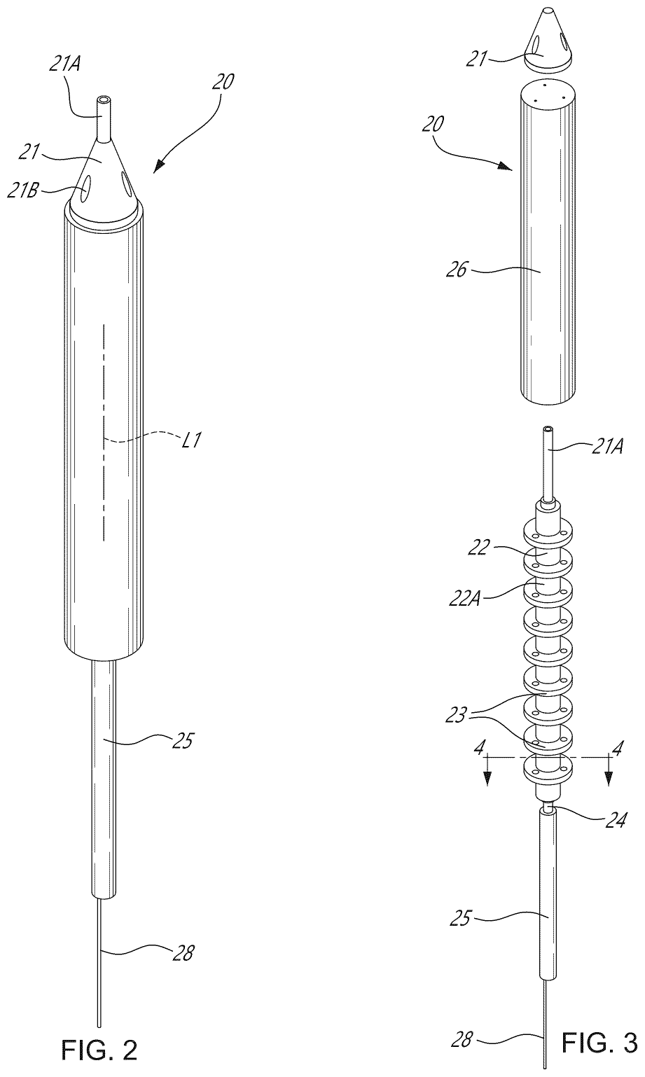

Referring to FIG. 4, a rib 23 of the ribs defines three or more guiding apertures 23B circumferentially distributed around the longitudinal axis L1. As illustrated, the central aperture 23A of the ribs 23 via which the central conduit 22 is inserted is separated from the guiding apertures 23B. They may be spaced apart from one another by 120 degrees. The guiding apertures 23B are used to receive cables 27, which may be nylon sutures, therethrough for actuation of the printing head 20 as will be described below. Therefore, the printing head 20 may include four channels: a central one defined by the central conduit 22 and three other ones defined by the guiding apertures 23B. The central conduit 22 may define a passage of about 1.27 mm in diameter and is used to flow hydrogels, in some embodiments. The guiding apertures 23B may have a diameter of about 0.72 mm and may be located at a radial distance of about 2.3 mm from the longitudinal axis L1. The guiding apertures 23B house the cables 27, which may be nylon sutures (e.g., F2448H, Ethilon), but any suitable cables may be used. It will be appreciated that the dimensions mentioned herein may be different without departing from the scope of the present disclosure. The guiding apertures 23B are in register with the nozzle apertures 21B to allow insertion of the cables 27 as will be described further below. Ends of the cables 27 may be secured to the nozzle 21.

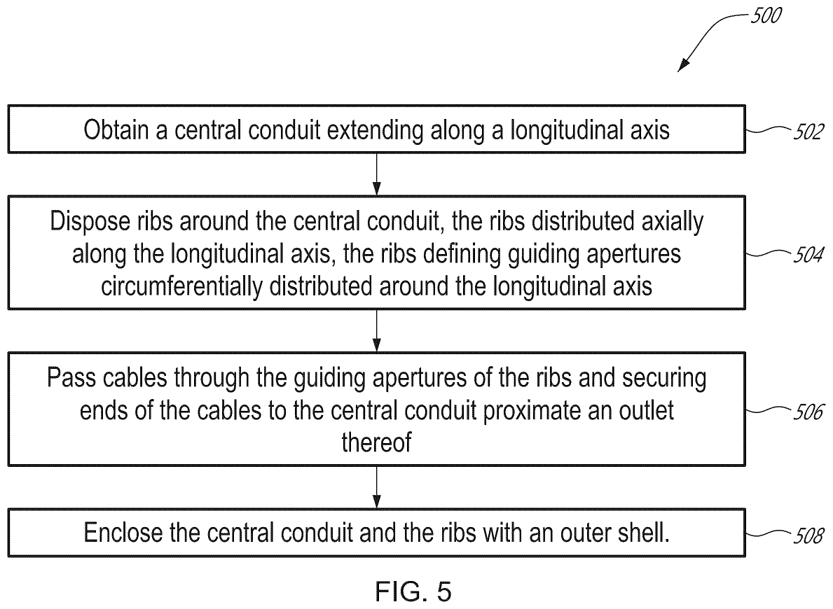

As shown in FIGS. 2-3, a rod 28 extends through the guiding apertures 23B and nozzle apertures 21B. The rod 28 is used for a manufacturing process of the printing head 20. More detail about the manufacturing process are presented below with reference to FIGS. 6A-6K. This rod 28 is removed at the end of the manufacturing process. The central aperture communicates with the guiding apertures.

Referring back to FIG. 1A, the actuation system 11 may include five actuators 12, which may be stepper motors, but any suitable actuators may be used. Two of those stepper motors are connected to the feeding tube 25 through gears to perform movements along a direction Z. The other three stepper motors may be in a main housing. The stepper motors may be connected to pulleys, which are connected to each respective cables 27 of the printing head 20. These actuators 12 are configured to vary a tension in one or more of the cables 27 relative to a remainder of the cable 27 to bend the central conduit 22 of the printing head 20. Actuation of the pulleys results in the shortening of the cables 27 within the printing head 20, leading to deformation and movements along directions Z and Y. In all directions, linear actuation is used to oppose for the Z-directional movement of the printing head 20. The actuator housing was designed for laryngoscopy, focusing on an in situ bioprinting method for the vocal folds. The actuators 12 may each individually connect to a driver, connected to a controller, such as an Arduino, in a separate housing. The separate housing may form an interface between the printing head 20 and user input from the input device 13, which may include a joystick, switch, and push buttons to setup and manipulate the printing head 20. In some embodiments, the controller may be a gaming controller (e.g., an Xbox™ or Playstation™ controller, or the like). Separating the electronics into two distinct boxes may enable a portable box with user inputs, while having the remainder of the electronics stationary and outside of the workspace of the surgeon.

Stated differently, cable-driven actuation may be used for the control and manipulation of the printing head 20. During actuation, the length of one or two of the cables decreased, resulting in the silicone base deforming. A hand-held casing was designed to house the actuators 12 (e.g., stepper motors). An electronics box was designed to house the drivers, microcontroller board (e.g., Arduino Mega 2560, Arduino), and remaining hardware. A physical control box was designed to maintain portable control. Control of the stepper motors was achieved by using the derived kinematic model to determine the required actuation based on user input. User input was achieved both by predetermined printing paths and manual control using a joystick. A kinematic model was derived to determine the position of the tip based on the actuation. The kinematic model assumed a constant curvature of the printhead. The second assumption was that only two cables may be actuated at any given time. The fixed parameters in the model are the distance of the cables to the center was denoted by r, and the angle of each cable relative to the z-axis, denoted by φ1, φ2, φ3. The actuation input to the printhead were the cable lengths, denoted by I1, I2, I3, controlled by the stepper motors. The configuration of the printhead was defined by its radius of curvature R, the arc-angle θ, and the angle relative to the z-axis φ. The task space was defined as the tip location in Euler coordinates denoted by Tx, Ty, Tz. The mappings of the printing head 20 is shown below:

$$q = {[ {l}_{1} , {l}_{2} , {l}_{3} ]}^{T} , {l}_{1} ⋁ {l}_{2} ⋁ {l}_{3} = 0 = {l}_{fixed} \qquad ( 1 )$$

$$C = {[ r , θ , φ ]}^{T} \qquad ( 2 )$$

$$T = {[ {T}_{x} , {T}_{y} , {T}_{z} ]}^{T} \qquad ( 3 )$$

During control of the endoscopic printhead, control was used to ensure one of the cables was always at its original length. Using one fixed cables, the cable lengths were translated into highest, lowest and fixed amount of actuation, denoted by I_{highest}, I_{lowest}, and I_{fixed}, as well as the angle of the cables relative to the z-axis, denoted by φ_{highest}, φ_{fixed}. Under the above assumptions and translation, the mapping from the actuator space and configuration space was done by:

$${φ}_{a} = {φ}_{highest} + \frac{π}{3} * \frac{{l}_{lowest}}{{l}_{highest}} \qquad ( 4 )$$

$$D = r * cos ( {φ}_{highest} - {φ}_{a} ) - r * cos ( {φ}_{fixed} - {φ}_{a} ) \qquad ( 5 )$$

$$θ = \frac{{l}_{fixed} - {l}_{highest}}{D} \qquad ( 6 )$$

$$R = \frac{{l}_{fixed}}{θ} \qquad ( 7 )$$

The mapping of the configuration space to the task space was done by:

$${T}_{x} = ( R + {D}_{c} ) * cos ( {φ}_{a} ) * cos ( θ ) - ( R + {D}_{c} ) * cos ( {φ}_{a} ) \qquad ( 8 )$$

$${T}_{Y} = ( R + {D}_{c} ) * sin ( {φ}_{a} ) * cos ( θ ) - ( R + {D}_{c} ) * cos ( {φ}_{a} ) \qquad ( 9 )$$

$${T}_{Z} = ( R + {D}_{c} ) * sin ( θ ) \qquad ( 10 )$$

For manual control, the configuration space was used as input for the control of the manipulator. During printing, a joystick was used to direct convert user-input into φₐ with a dummy actuation length denoted by I_{dummy}. Based on the φₐ, the cable number of the fixed cable was determined and denoted by φ_{fixed}. The corresponding configuration space was determined by:

$$θ = \frac{{l}_{fixed} - {l}_{dummy}}{- r * cos ( {φ}_{fixed} - {φ}_{a} ) + r} \qquad ( 11 )$$

$$R = \frac{{l}_{fixed}}{θ} \qquad ( 12 )$$

Under the assumptions for the initial mapping the mapping of the configuration space to the task space was done for each cable by:

$$D = r * cos ( {φ}_{1 , 2 , 3} - {φ}_{a} ) - r * cos ( {φ}_{fixed} - {φ}_{a} ) \qquad ( 13 )$$

$${l}_{1 , 2 , 3} = θ * ( R - D ) \qquad ( 14 )$$

During control of the manipulator first the local coordinates and required length of each stepper motor are calculated. Based on the longitudinal movement obtained in eq 10, the steps required for longitudinal movement are calculated. The longitudinal movement is subtracted from the required actuation in the cables to maintain tension. Lastly, all stepper-motors are actuated simultaneously, all actuation is spaced evenly over a 150 millisecond period, in order to achieve smooth motion.

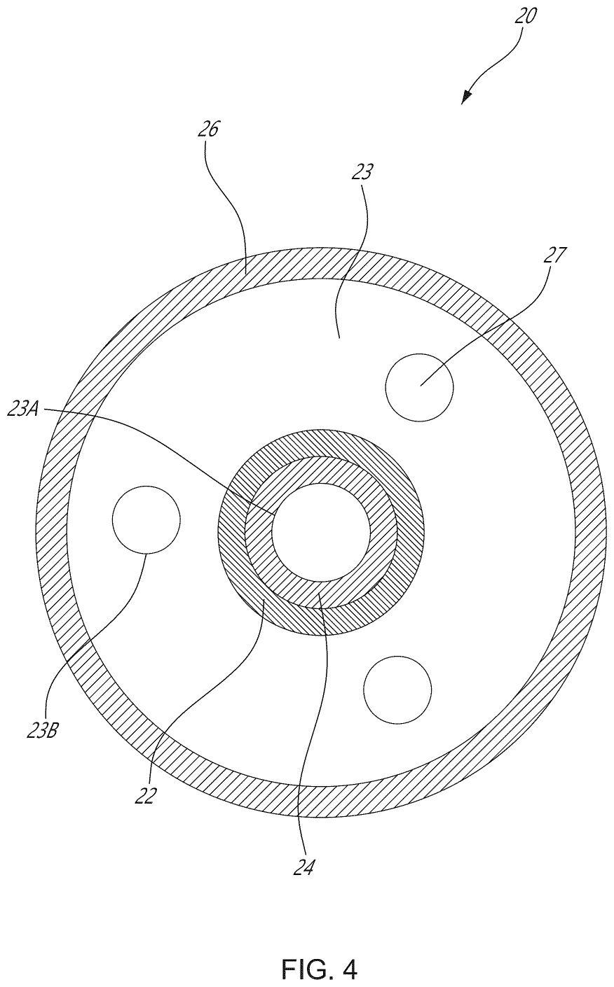

Referring now to FIG. 5, a method of manufacturing the printing head 20 is shown at 500. The method 500 includes obtaining the central conduit 22 at 502; disposing the ribs 23 around the central conduit 22 at 504; passing the cables 27 through the guiding apertures 23B of the ribs 23 and securing ends of the cables 27 to the central conduit 22 proximate an outlet thereof at 506; and enclosing the central conduit 22 and the ribs 23 with the outer shell 26 at 508.

In some embodiments, the obtaining of the central conduit 22 includes obtaining the central conduit having the nozzle 21 secured thereto at the outlet of the central conduit 22. The method may alternatively include securing the nozzle 21 to the outlet of the central conduit 22.

In the embodiment shown, the securing of the ends of the cables 27 to the central conduit 22 includes securing the ends of the cables 27 to the nozzle 21 such that the cables 27 are secured to the central conduit 22 via the nozzle 21.

The disposing of the ribs 23 around the central conduit 22 includes inserting the central conduit 22 through central apertures defined by the ribs 23. In the embodiment of FIG. 2, the method includes disposing each of the ribs 23 within a respective one of the annular grooves 22A grooves distributed longitudinally on the central conduit 22 along the longitudinal axis L.

In some embodiments, the method includes inserting tubes into the guiding apertures. The passing of the cables through the guiding apertures includes passing the cables into the tubes. The method then includes removing the tube after the injecting of the elastic material into the second mold.

Referring to FIGS. 6A to 6K, the obtaining of the central conduit 22 may include molding the central conduit 22 as will be discussed further below. The molding of the central conduit 22 may include: disposing a rod inside a first mold having a shape corresponding to that of the central conduit; injecting an elastic material in the mold and around the central conduit; and removing the rod to define a central passage of the central conduit. In the embodiments shown, the enclosing of the central conduit 22 and the ribs 23, 132 with the outer shell 26 includes: disposing the central conduit and the ribs in a second mold after the passing of the cables through the guiding apertures; and injecting the elastic material into the second mold thereby creating the outer shell and filling spaces defined between the ribs.

More specifically, to make the printing head 20, a two-cast process may be used using two distinct molds. The molds and resin parts may be printed using a stereolithography printer, although any suitable process may be used to obtain the molds. The photo-initiator in the resin prevented the silicone from curing. To ensure curing, the molds and resin parts were washed with ethyl alcohol. The parts were then submerged in distilled water and post-cured for 30 minutes in a curing station. After post-curing the molds were kept submerged at room temperature for 24 hours. The post-curing step may be repeated 5 times, replacing the water between washes.

Referring to FIG. 6A, after washing, a 100 mm, 18-gauge cut-to-length, stainless steel tube 50, with a resin flange 50A placed over it, was placed in the center of a first mold 51. The first mold 51 was closed and sealed using screws. Then, a quantity (e.g., 5 gram) of a first silicone (e.g., Silicone 00-50, Smooth-on) was mixed with a quantity (e.g., 5 gram) of a second silicone (e.g., silicone 00-50). The silicone mixture was then placed in a vacuum chamber for 15 minutes to degas the mixture. After degassing, the mixture was transferred to a 10 ml syringe and injected into the mold until silicone poured from the venting hole. The silicone was cured for 24 hours at room temperature. As shown in FIG. 6B, the resultant was removed from the first mold 51 to yield the central conduit 22. As shown in FIG. 6C, the ribs 23 were installed on the central conduit 22.

As shown in FIG. 6D, the nozzle 21 is mounted to the central conduit 22 with the tip 21A, which may be a 15 mm, 18 gauge cut-to-length, stainless steel tube. Loctite™ or other fastening means may be used to secured the nozzle 21 to the central conduit 22 and the tip 21A to the nozzle 21. A 150 mm cut-to-length, 22-gauge stainless steel tube 52, was guided through the tip 21A. The excess silicone layer was removed from the central conduit 22. As illustrated in FIG. 6E, three stainless steel tubes 53 were guided through the nozzle apertures 21B and guiding apertures 23B of the ribs 23 and used to align the ribs 23. As shown in FIG. 6F, the cables 27 guided through the tubes 53.

Referring to FIG. 6G, the assembly was then placed in a second mold 54 and closed using screws. A quantity (e.g., 10 gram) of a silicone mixture was prepared and injected as described before. After 24 hours, the assembly was removed from the second mold 54 (FIG. 6H). The stainless steel tubes 53 were carefully removed from the assembly (FIG. 6I). The cables 27 are then fixed to the nozzle 21 using Loctite™ or other fastening means and excess cables 27 were cut off after 5 minutes (FIG. 6J).

As shown in FIG. 6K, a 20 mm, 18-gauge cut to length, the tube 24, which may be a stainless steel tube, was inserted for 5 mm at the base and fixated using Loctite™ or other fastening means. After the Loctite™ was set, excess silicone was removed and the feeding tube 25 was placed over the tube 24 and fixated using Loctite™ or other fastening mean to complete the manufacturing.

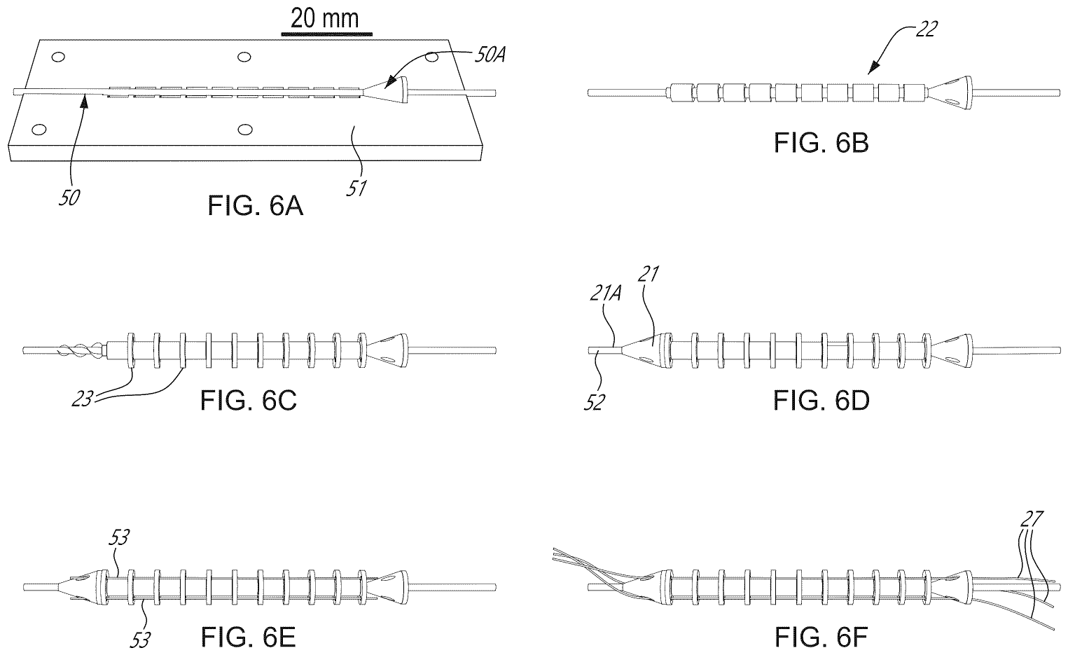

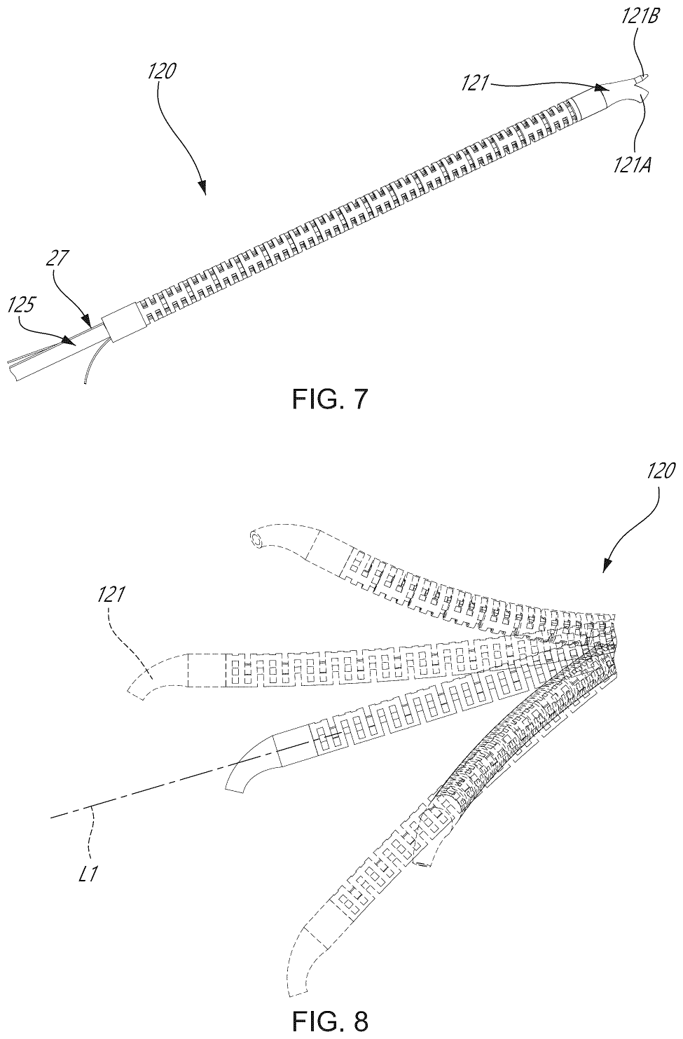

Referring to FIGS. 7-11, the printing head 120 is described in more detail. The printing head 120 differs from the printing head 20 described above with reference to FIGS. 2-4 by the ribs 23. More specifically, the printing head 120 includes a core 130. The core 130 may be a single monolithic body. The core 130 defines a central passage 131 via which a central conduit may be received. The core 130 includes ribs 132 spaced apart from one another longitudinally. The ribs 132 are interconnected to one another via longitudinal members 133. Each of the longitudinal members 133 interconnects two axially adjacent ones of the ribs 132. In the embodiment shown, the longitudinal members 133 are circumferentially offset from one another along the longitudinal axis L1 of the core 130. Put differently, circumferential positions of the longitudinal members 133 vary along the longitudinal axis L1.

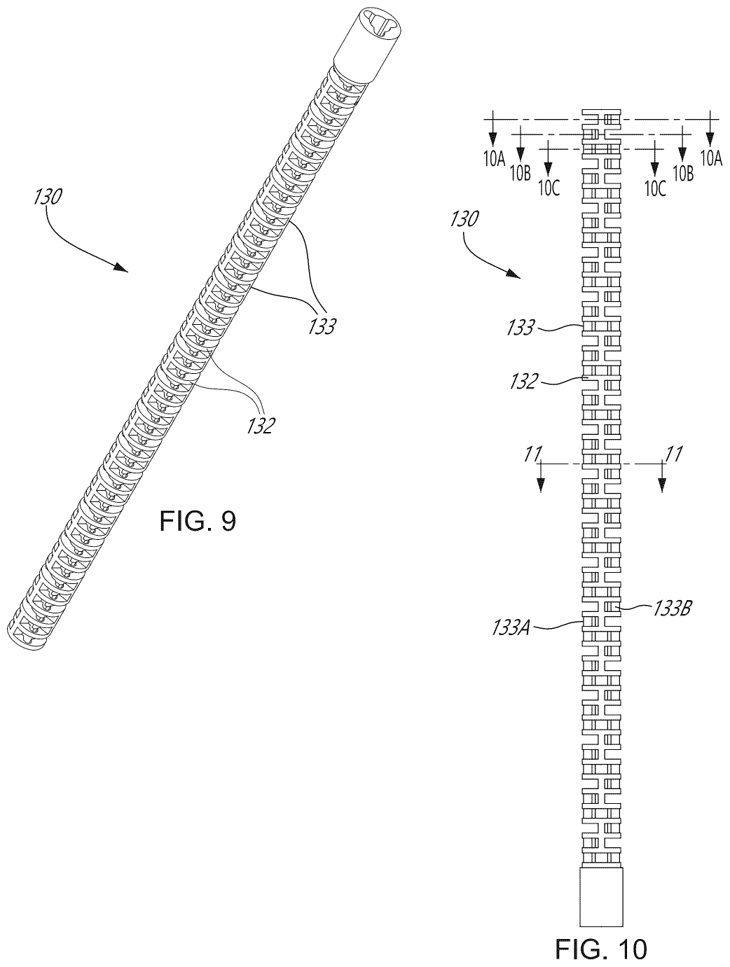

Referring to FIGS. 10A to 10C, in the embodiment shown, each two adjacent ones of the ribs 132 are interconnected to one another via two ribs 133A, 133B that are circumferentially offset from one another by about 120 degrees, although a different offset may be used. A repeating pattern is shown in FIGS. 10A to 10C for three consecutive spaces defined between four consecutives ribs 132. As illustrated, the longitudinal member 133B of FIG. 10A is in register with the longitudinal member 133B of FIG. 10B while the two longitudinal members 133A are offset from one another by 120 degrees. The longitudinal member 133B of FIG. 10B is circumferentially between the longitudinal members 133A, 133B of FIG. 10C and the longitudinal members 133A, 133B of FIG. 10B are both offset from the longitudinal members 133A, 133B of FIG. 10C. They may be free from circumferential overlap. In other words, the longitudinal members include a pair of longitudinal members between a first rib and a second rib of the ribs. The longitudinal members of the pair are circumferentially offset from one another (e.g., by about 120 degrees). The pair of the longitudinal members are clocked relative to a second pair of the longitudinal members disposed between the second rib and a third rib. Then, the pattern repeats itself for the subsequent ribs 132. This configuration may provide the required flexibility to the core 130. Other pattern are considered.

The core 130 may be made of a resin polymer, which may be stiff. The flexibility of the core 130 may be imparted by the segmentation created by the longitudinal members 133 that interconnect the ribs 132 to one another as described above.

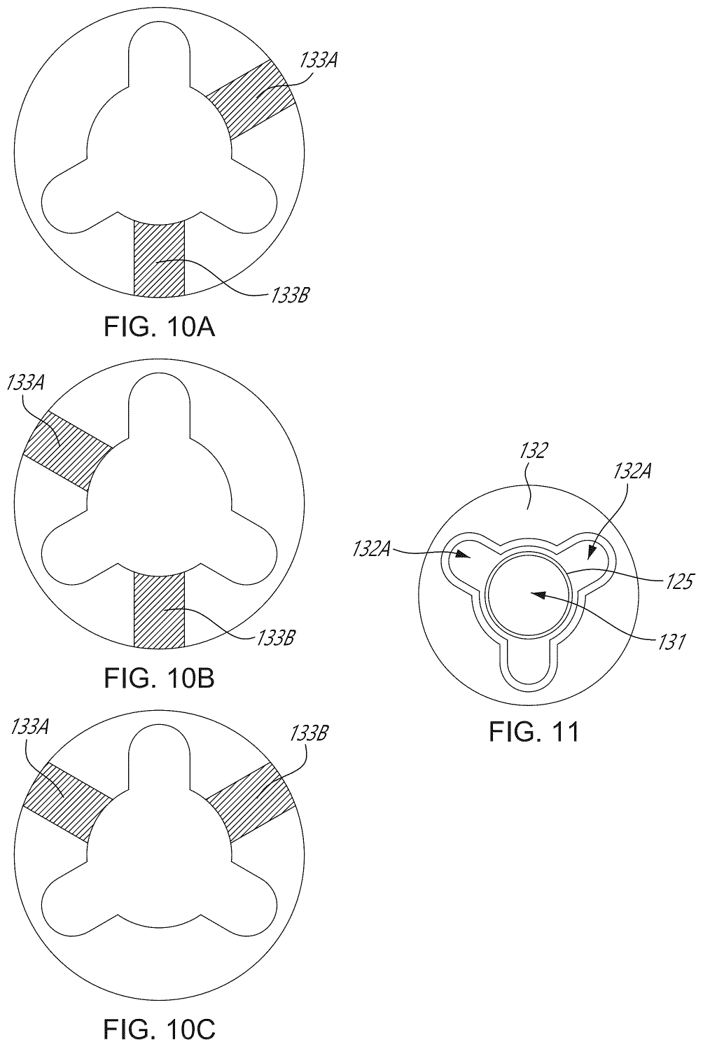

The core 130 may be manufactured using a molding process as described above with reference to FIGS. 6A-6K. Alternatively, the printing head 120 may be made with additive manufacturing. As shown in FIG. 11, the ribs 132 define apertures 132A for receiving the cables 27 as described above. In this embodiment, the apertures 132A communicate with the central passage 131. This may allow a diameter of the head 120 to reach about 2.7 mm. This may result in radial gaps between outer peripheries of the ribs 132 and the apertures 132A to be about 200 microns. The cables 27 that are routed through the apertures 132A may be isolated from the central passage 131 via a tube 125, which may be made of any suitable material able to withstand a pressure exerted by the hydrogel and allowing flexibility of the head 120.

Referring back to FIG. 7, the printing head 120 has a nozzle 121 that may be arched such that an outlet 121A thereof has a component along a radial direction relative to the longitudinal axis L1. The outlet 121A may thus be radially offset from the longitudinal axis L1. This may facilitate the printing of the vocal folds. However, other nozzles may be used depending of the intended application of the head 120. Any suitable nozzle may be used without departing from the scope of the present disclosure. The nozzle 121 may further include a tip 121B, which may be in register with the longitudinal axis L1. The tip, which may be sharp, may be used for placement of a bead (e.g., Styrofoam™ bead), which may be used for position tracking. This is however optional.

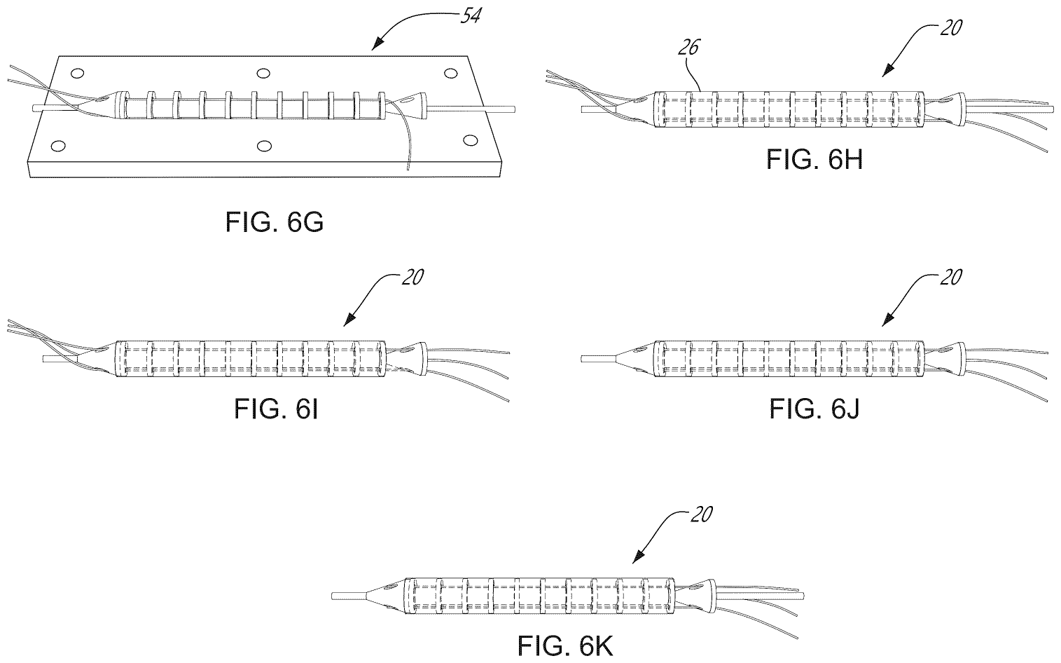

Referring to FIG. 8, a plurality of possible positions of the printing head 120 are shown. As illustrated, the head 120 may move such that the nozzle 121 moves radially and axially relative to the longitudinal axis L1. This may allow the printing head 120 to print complex structures in restricted locations (e.g., vocal folds).

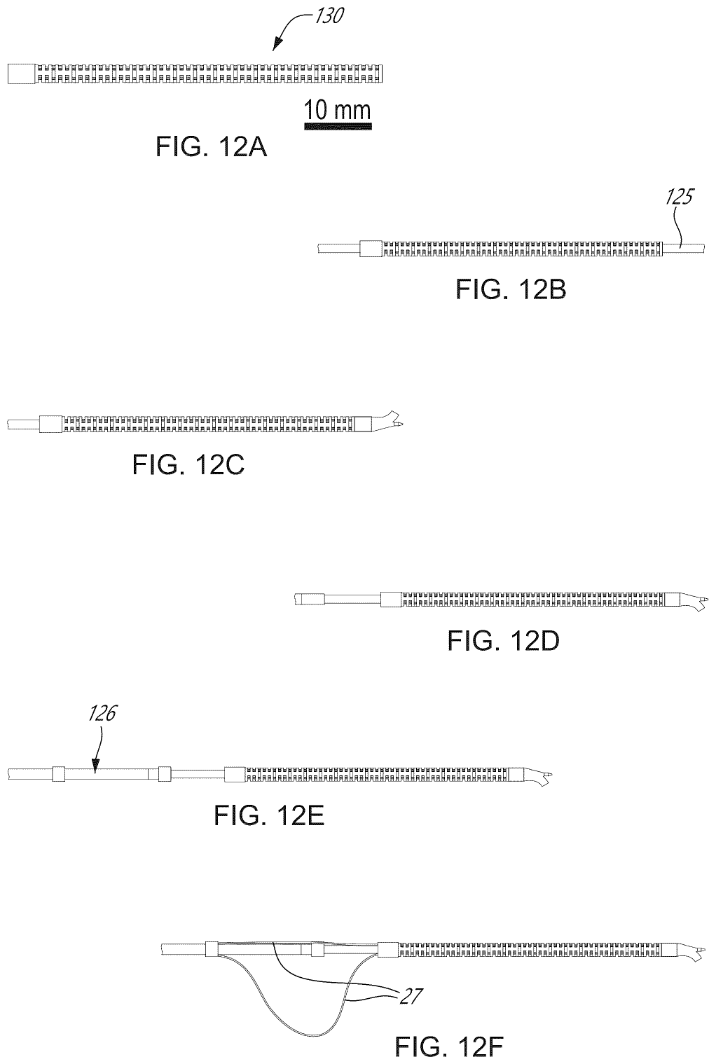

Referring now to FIGS. 12A to 12F, a method of manufacturing the printing head 120 is described. The head 120 is manufactured with an additive manufacturing process. The process may include preparing the core 130 and nozzle 121 for additive manufacturing with support structures manually positioned at the base along the axial direction using a 0.3 mm touch point diameter to minimize surface artifacts. The components may be fabricated using a stereolithography printer (e.g., Form 3, FormLabs, USA) with a photopolymer resin (e.g., Grey V4, FormLabs, USA). The components may be cleaned with a 30 minute wash cycle in 99.5% isopropyl alcohol in a washing station. Post-curing of the components may be performed for 60 minutes at 60° C. in a curing station (e.g., Form Cure 2nd generation, FormLabs, USA).

The printing head 120 shown in FIG. 12A may be fabricated by inserting a primary polymer tube 125 into the core 130 as shown in FIG. 12B. The nozzle 121 may be placed at the distal end and secured (e.g., glued, fastened, etc) as shown in FIG. 12C. The glue may be cured at room temperature for 10 minutes. The polymer tube 125 may then be cut to length with an excess of 17 mm at the base as shown in FIG. 12D. A secondary polymer tube 126 may be positioned over the tube 125 for 4 mm, as shown in FIG. 12E. Cable guides 27 were positioned on the secondary tubing, as shown in FIG. 12F and routed through the apertures as described above. Then, the needles on three nylon sutures (Ethicon 669H, Ethilon, USA) were removed. The sutures were used as cables and were guided through the cable guides and inserted in the flexible backbone. The cables may then be fixed in place at the nozzle 121. The completed printhead was inserted into the stainless steel tube 19 (FIG. 1A) and secured via a press-fit connection. Finally, the cables were connected to their respective drums (FIG. 1A).

### Data-Driven Kinematic Model

Control of the endoscopic printhead is achieved by varying the cable lengths and the linear stepper motor, leading to the deformation of the flexible arm. A data-driven kinematic model was used to derive the relationships between the cable actuations and nozzle locations, reducing required assumptions and complexity. The used parameters a length of the three cables 27 l_{0,i}, i∈{1,2,3}. At each time step, t, the length of the cables is changed, relative to the initial length by Δl_{t,i}. On the robot all admissible positions have at least one cable at its initial length, Δl_{0,i}, which is denoted by l_{t,u}. The radius of the cables to the axial centerline is denoted by r. Given the changes in length, the actuations are categorized as: 1) The primary cable actuation, Δl_{t,p}, which is defined by the highest prescribed actuation value. 2) The secondary cable actuation, Δl_{t,s}, which is the cable with actuation equal or lower than the primary cable. 3) The unchanged cable, Δl_{t,u}, where the cable is at the original length. The corresponding orientation of the primary, secondary and unchanged cable is denoted by ϕₚ, ϕₛ, and ϕᵤ. Using the ratio between the primary and secondary cable actuations, the bending direction of the printhead, ϕ, can be calculated with (Equation 1). The distance, Dᵤ, between l_{t,u} and the centerline parallel to the bending orientation can be calculated with (Equation 2).

$$ϕ = {ϕ}_{p} \pm \frac{π}{3} \cdot \frac{Δ {l}_{t , s}}{Δ {l}_{t , p}} \qquad ( Equation 1 )$$

$${D}_{u} = - r \cdot cos ( {ϕ}_{u} - ϕ ) \qquad ( Equation 2 )$$

The tip displacement vector in the xy-plane, \vec{XY}, is linearly dependent on the sum of all cable actuations, Δl_{T}, and Dᵤ with the factor a, and is calculated with (Equation 3) and (Equation 4): Factor a is empirically derived.

$$Δ {l}_{T} = \sum Δ {l}_{t , i} \qquad ( Equation 3 )$$

$$\vec{XY} = \frac{Δ {l}_{T} \cdot a}{{D}_{u}} \qquad ( Equation 4 )$$

The displacement in the z-direction has a parabolic relationship with the displacement vector with the factors b, c and d, which are empirically derived. Using the displacement vector and the bending orientation the cartesian coordinates can then be calculated according to (Equation 5), (Equation 6), and (Equation 7).

$${T}_{x} = \vec{XY} \cdot cos ( ϕ ) \qquad ( Equation 5 )$$

$${T}_{y} = \vec{XY} \cdot sin ( ϕ ) \qquad ( Equation 6 )$$

$${T}_{z} = b \cdot {T}_{x} = {\vec{XY}}^{2} + c \cdot \vec{XY} + d \qquad ( Equation 7 )$$

Control of the nozzle position is achieved by translating target coordinates into required actuation of each cable to reach the target coordinates. The joystick input prescribes an incremental directional change in position. From the new target position the displacement vector and bending direction is calculated using (Equation 8) and (Equation 9).

$$\vec{XY} = \sqrt{{T}_{x}^{2} + {T}_{y}^{2}} \qquad ( Equation 8 )$$

$$ϕ = {cos}^{- 1} \frac{{T}_{x}}{\vec{XY}} \qquad ( Equation 9 )$$

Given the bending direction and displacement vector, the corresponding cable actuations are calculated. From the bending direction, the cable categorization is determined. The categorization is based on the difference between the cable orientation and bending direction where: 1) The lowest difference corresponds to the primary cable. 2) The largest difference corresponds to the unchanged cable. 3) The remaining angle corresponds to the secondary cable. The sum of all cable actuations is calculated with (Equation 4). The ratio, Rₐ, between the primary and secondary cable actuations is calculated by rewriting (Equation 1) into (Equation 10). The corresponding primary and secondary cable actuations are then obtained with (Equation 11) and (Equation 12). The change in the axial direction is calculated according to (Equation 7) with the new target position.

$$\frac{Δ {l}_{t , s}}{Δ {l}_{t , p}} = {R}_{a} = \pm ( ϕ - {ϕ}_{p} ) \cdot \frac{3}{π} \qquad ( Equation 10 )$$

$$Δ {l}_{t , h} = \frac{Δ {l}_{T}}{{R}_{a} + 1} \qquad ( Equation 11 )$$

$$Δ {l}_{t , l} = Δ {l}_{t , h} \cdot {R}_{a} \qquad ( Equation 12 )$$

To direct the nozzle to the target position, the change in cable lengths and axial position are calculated relative to the previous time step. The change in lengths is calculated as steps required for each stepper motor as well as for the linear stepper motor to compensate for bending of the printhead. To obtain linear motion from current point to target point, the steps for each stepper motor are evenly distributed over a time step of 30 ms.

### Results for the Printhead 20

Two different hydrogels were tested for printability. A Pluronic F217 hydrogel and Alginate hydrogel. Pluronic F217 (Sigma-Aldrich, New South Wales, Australia) was dissolved in distilled water using a 19% w/v ratio. The solution was kept at 4° C. for 24 hours. After dissolving, 0.001% Toluidine blue (Sigma-Aldrich, New South Wales, Australia) was added to the solution for visualization. Alginate (I-1G, Kimica, Tokyo, Japan) was dissolved in 3% w/v ratio in distilled water and 0.001% Toluidine blue was added. Calcium chloride (Sigma-Aldrich, New South Wales, Australia) was dissolved in distilled water to create a 50 Mm solution. The 1.5% alginate hydrogel was formed by mixing the 3% alginate solution with the 50 Mm calcium chloride solution in a 1:1 ratio.

The printability of both hydrogels was tested by printing single lines on a hot plate of 37° C. with a fixed writing speed of 2 mm/s using different pressures for the pneumatic dispensing. The printability was determined by calculating the average extrusion diameter and visual analyses of the ink. The average extrusion diameter was obtained by measuring the width of each line at 5 random locations using image processing (1.53 T, ImageJ). Visual analyses was performed to identify whether the line was created continuously or whether the lines were interrupted.

To demonstrate the three-dimensional printability capabilities of the endoscopic printhead, different shapes were printed using manual control of the printhead. The handheld housing was placed on a concealed box with a 35 mm orifice. The endoscopic printhead and rigid catheter were guided through the orifice and moved to the target location on the heating bed set at 37° C.

Construct were printed using the writing speed of 2 mm/s and pressures determined during the printability test, 17.5 and 35 PSI for the alginate and Pluronic hydrogels respectively. Manual constructs were printed in two different configurations, single layer and multilayer with both hydrogels. The single layer constructs consisted of a spiral, circular, triangular and squared shape. Multilayer constructs consisted of squared, triangular and M-shaped constructs repeating build using 4 layers. The endoscopic printhead was moved in the z-direction for each layer. Each task was repeated five times for practice before making a final construct. Robotic prints were created, using the alginate hydrogel, by using predetermined printing paths. The printing paths consisted of vectors of movement to create the respective shapes, instead of the joystick input. The printing paths consisted of square, star, circular, S-shaped, and triangular shapes. The path was repeated 5 times, moving the printhead for 1 mm in the z-direction between each layer.

Three manipulators were used for position tracking measurements. Different levels of pretension were investigated. The error of the measured position did not change significantly between 5, 10, 12.5 and 15%. The mean error reduced from 3.88 to 0.35 mm when increasing the pretension from 0 to 10%. The variance between the measurements was significantly different for all groups compared to 10% pretension. Both the variance between manipulators and the RMS error were lowest at 10% pretension. For this reason, 10% pretension was used for further tests. Reliability testing were performed. The RMS error increased from 0.12 mm to 0.15 mm after 10 cycles. The increase in error was not significant. The variance between cycles is significant, caused by drifting of the manipulator in one of the measurements. The manipulator that drifted during different cycles had an error of 0.2 mm after 10 cycles. The total error of the identified manipulator was 0.50 which was accepted as accurate for accurate control of the manipulator for printing. Hysteresis was also testing. No significant changes were found between 0.05 seconds and 1 minute after actuation, indicating that there was no effect of hysteresis on actuator movement.

Two different hydrogels were used for printability measurements. It was observed that the average filament diameter of the alginate hydrogel did not increase significantly at increased pressures. At pressures below 20 PSI, there was no continuous deposition of ink leading to gaps. At increased pressures, the thickness of the filament varied, while printing of alginate below 15 PSI resulted in inconsistent deposition. The optimal pressure for printing of Alginate was 17.5 PSI, with a resolution of 1.6 mm, lowest variation and consistent deposition. Printing of Pluronic F217 below 35 PSI resulted in inconsistent deposition. The optimal pressure for printing of Pluronic F217 was 35 PSI, with the a resolution of 1.5 mm, lowest variation and consistent deposition.

The alginate and Pluronic F217 hydrogels were used to demonstrate the three-dimensional printing capabilities of the endoscopic printhead. Four-layered circles, triangles and letters were printed. Each layer was completed in a single stroke. Corrections were made in case of inconsistent extrusion.

### Results for the Printhead 120

The endoscopic printhead and kinematic model were evaluated using optical position tracking. Evaluation was done for accuracy, repeatability and position drift of the nozzle tip position. The accuracy of the printhead refers to the ability to precisely predict the nozzle tip position. Repeatability is the ability of the printhead to repeat the same pattern over multiple cycles. The position drift is the ability of the printhead to maintain a fixed nozzle position over time at a specified actuation. The characterization was performed on six different printheads. The position error, E, was the distance between the target position and measured position. A calibration step was performed prior to using each printhead to straighten it and determine the cable orientation.

It was observed that the accuracy of the printhead and kinematic model across 340 cable actuation combinations, varying incrementally from 0 and 2.76 mm for two cables, with one cable remaining at the original length. The error increased with increased nozzle tip displacement. The accuracy was evaluated at a restricted workspace, with a maximal nozzle tip displacement of 10 mm. Consistent performance across ten measurement cycles has been observed, with printhead 3 and 6 indicating minor variation after the initial ten cycles. The error varied between individual printheads with an average error within the workspace of 1.33 mm across all printheads and cycles.

The endoscopic printhead demonstrated high repeatability to reproduce a squared motion pattern. The position error of the printhead during the first cycle showed comparable results to the accuracy measurements. The subsequent cycles had a maximal deviation of 0.25 mm relative to the positions in the first cycle.

The printhead showed a stable position over time for most measured positions. The initial nozzle position error was below 0.4 mm and gradually decreased for 19 out of 24 measured nozzle tip positions. The remaining five measured nozzle tip positions had an initial error below 0.8 mm. The five positions were at measurement at prescribed position 2 and 4, associated with upward and downward actuation.

The printability of the hydrogel is dependent on the supply pressure with a fixed writing speed and nozzle geometry. Printability was assessed by measuring the resolution, which is the width of printed lines. Measurements were performed using a dopamine-grafted hyaluronic acid with silk fibroin hydrogel (DAHA). The DAHA hydrogel was used. The mean printed line width increased at higher pressures due to increased flowrates of the hydrogel. At lower pressures, inconsistent flow resulted in interrupted lines. The printing homogeneity was assessed by normalizing the standard deviation against the mean widths. Increased supply pressures lead to a slight decrease in homogeneity. The optimal consistent printing with a resolution of 1.2 was achieved at 262 kPa with a mean line with of 1.2 mm. The supply pressure was set to 262 kPa in subsequent measurements.

The functionality of the MIISB was demonstrated by printing constructs on a flat surface as well as an ex-vivo surgical model. The constructs created demonstrate the ability of the in-situ bioprinter to control deposition of hydrogel and create three-dimensional features on a flat surface. A single layer constructs was obtained and shows increased complexity of structures relative to the multilayer constructs. The decreased complexity of multilayer constructs is due to increased difficulty with handheld control to reproduce the same complex pattern consistently. The multilayer constructs demonstrate the ability to control deposition and create simple geometric constructs.

The MIISB may offer the ability to successfully restore vocal fold geometry with accurate hydrogel deposition in surgical ex-vivo models. The clinically relevant abnormalities were sulcus and a lesion. Additionally, a fully removed vocal fold was treated. Reconstruction was done using a simulated suspension laryngoscopy environment. The sulcus and lesion model were treated, and the vocal fold geometry was successfully restored. The full vocal fold removal further demonstrated the ability to accurately control the deposition of the hydrogel.

### CONCLUSION

Single-layer and four-layer constructs, using manual control, and five layer-constructs were created with robotic printing. The results show the ability for accurate control and real-time adaptation. The use of a continuum robot design shows promise for enabling minimally invasive procedures through a natural orifice or small incision.

In this disclosure, an endoscopic printhead for in situ bioprinting using a cable-driven continuum robot design is presented. The actuation using cables may reduce complexity, which increases the possibility of scalability, shown by the 8 mm diameter of the design. Accurate and real-time control is obtainable with the disclosed print head for manual printing, which was accomplished by deriving a kinematic model with low computational power. The average error of the proposed kinematic model was 0.35 mm at 10% pre-tension. At lower levels of pretension, the error significantly increased. This is expected due to non-homogenous stresses when lower levels of pretension are used. Other designs for minimally invasive surgery have reported similar errors and are acceptable for accurate movement. The use of pretension removes the possibility of slack cables.

The measured errors are within 100 micron and there was no change in error over time. The characterization was done within the workspace established. The established workspace has a reachable length of 13 mm, which is sufficient for the use in vocal folds. The average length of the vocal folds in male is 14 mm, and can increase to 20 mm in professional singers. The expected use for in situ bioprinting is the reconstruction of vocal fold after the removal of a lesions which are typically smaller than the vocal folds based on images from surgical procedures.

The printability and resolution of hydrogels depend on the nozzle diameter, writing speed, pressure, and mechanical properties of the hydrogel. Since the proposed printhead may be used with manual control, the writing speed of the endoscopic printhead was fixed to allow for accurate control. The researched hydrogels were chosen to investigate the ability of the endoscopic printhead to print different types of hydrogel. The crosslinking of Pluronic F217 is temperature-dependent and reversible. Alginate hydrogels are shear thinning. Rheological characterization was performed to optimize the solidification temperature of Pluronic F217 and printability of the alginate hydrogel. As a result of the molecular weight of alginate, the required concentrations may alter. The effect of pressure was investigated to create optimal printing settings with a fixed nozzle diameter (18 Gauge). The resolution of described continuum robot printers of hydrogels found a resolution achievable between 0.8 and 2 mm, which is comparable to the results described herein. The resolution of the printing process in pneumatic extrusion printing is dependent on a variety of parameters. Herein the focus lay on a fixed writing speed for accurate manual control and varying the pressures. The resolution is also dependent on the die-swelling behavior of the used ink and material properties. Increasing the resolution requires increasing the printing pressures. To improve the functionality of hydrogels for regenerative purposes, incorporating cells is preferable. Cell viability is decreased during extrusion-based printing due to shear stresses acting on the cells. The viability is dependent on the nozzle diameter, pressures, and bio-ink used.

The results show the ability to print both shear-thinning hydrogels and temperature-dependent hydrogel, thereby increasing the range of possibilities for regenerative properties.

This disclosure proposes a minimally invasive in-situ bioprinter designed for compatibility with suspension laryngoscopy, which may be used for other applications. The design may incorporate a 2.7 mm endoscopic printhead using cable-driven actuation. The printhead and vocal fold can be visualized using a standard microscope used during phonosurgery. The deposition location is manually controlled in real-time, reducing procedural complexity and need for pre-surgical planning of the defect. Pre-surgical planning may be compromised due to patient movement, surgical intervention and the scanning quality.

The MIISB may offer high spatial control, which may lead to precise hydrogel placement. The results of the MIISB may provide accurate position control with high repeatability and low position drift within a 20 mm workspace. This may allow for accurate hydrogel deposition on most vocal folds, which have a length of 14.9 to 20.9 mm. The accuracy of the printhead may be improved by filtering underperforming printheads after calibration. The supply pressure was optimized resulting in consistent homogenous hydrogel deposition with a line width of 1.2 mm. These results combined enable the use of the MIISB to reconstruct the vocal fold geometry in three different models. The injection of the ex-vivo models indicated the MIISB's capacity to restore the vocal fold geometry required for phonation.

The MIISB does not require external magnets for actuation and position control as described. It may be devoid of such magnets.

MIISB may be able to reduce limitations with current methods for hydrogel injections for vocal fold repair. With the improved hydrogel localization and distribution, the effectiveness of these hydrogels may improve and decrease the risk of post-surgical fibrosis. The potential reduction in post-surgical fibrosis and improve deposition may lead to improved quality of life for patients.

The disclosed devices and printheads may allow for accurate and repeatable printing. The printheads may be controlled via the input devices as described, such that the medical practitioner controls the movements of the nozzle. Alternatively, the controller may be preloaded with a determined path and the nozzle may follow this path automatically.

It is noted that various connections are set forth between elements in the preceding description and in the drawings. It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities. The term “connected” or “coupled to” may therefore include both direct coupling (in which two elements that are coupled to each other contact each other) and indirect coupling (in which at least one additional element is located between the two elements).

It is further noted that various method or process steps for embodiments of the present disclosure are described in the preceding description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various aspects of the present disclosure have been disclosed, it will be apparent to those of ordinary skill in the art that many more embodiments and implementations are possible within the scope of the present disclosure. For example, the present disclosure as described herein includes several aspects and embodiments that include particular features. Although these particular features may be described individually, it is within the scope of the present disclosure that some or all of these features may be combined with any one of the aspects and remain within the scope of the present disclosure. References to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. The use of the indefinite article “a” as used herein with reference to a particular element is intended to encompass “one or more” such elements, and similarly the use of the definite article “the” in reference to a particular element is not intended to exclude the possibility that multiple of such elements may be present.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. An endoscopic printing device comprising:
a printing head having:
a nozzle for outputting a biomaterial to be printed;
a central conduit extending along a longitudinal axis and fluidly connectable to a source of the biomaterial, the central conduit fluidly connected to the nozzle, the central conduit being flexible;
ribs disposed around the central conduit and spaced apart from one another along the longitudinal axis, a rib of the ribs defining three or more guiding apertures extending axially through the rib and being circumferentially distributed around the longitudinal axis, the ribs being interconnected to one another; and
cables extending through the guiding apertures, ends of the cables attached to the central conduit proximate the nozzle, the cables configured to be engaged to an actuator for controlling motion of the endoscopic printing head; and
an actuation system engaged to the cables, the actuation system including actuators each engaged to a respective one of the cables, the actuators operable to vary a tension in one or more of the cables relative to a remainder of the cable to bend the central conduit.

2. The endoscopic printing device of claim 1, wherein the ribs are interconnected to one another by longitudinal members extending axially relative to the longitudinal axis from one of the ribs to an adjacent one of the ribs.

3. The endoscopic printing device of claim 2, wherein the longitudinal members include a pair of longitudinal members between a first rib and a second rib of the ribs, longitudinal members of the pair being circumferentially offset from one another by about 120 degrees, the pair of the longitudinal members being clocked relative to a second pair of the longitudinal members disposed between the second rib and a third rib.

4. The endoscopic printing device of claim 1, wherein the ribs define central passages sized to receive the central conduit, the guiding apertures opening to the central passages.

5. The endoscopic printing device of claim 4, wherein the central conduit is a tube received through the central passages of the ribs, the cables being isolated from one another by a cooperation of the ribs and the tube.

6. The endoscopic printing device of claim 1, wherein the ribs are interconnected to one another by an outer shell extending around and secured to peripheries of the ribs.

7. The endoscopic printing device of claim 6, wherein the outer shell is made of an elastic material.

8. The endoscopic printing device of claim 6, wherein spaces defined between the outer shell, the central conduit, and the ribs are filled with an elastic material.

9. The endoscopic printing device of claim 6, wherein the central conduit is separated from the guiding apertures.

10. The endoscopic printing device of claim 1, wherein the ends of the cables are secured to the nozzle, the ends of the cables secured to the central conduit via the nozzle.

11. An endoscopic printing head for bioprinting, comprising:
a nozzle for outputting a biomaterial to be printed;
a central conduit extending along a longitudinal axis and fluidly connectable to a source of the biomaterial, the central conduit fluidly connected to the nozzle, the central conduit being flexible;
ribs disposed around the central conduit and spaced apart from one another along the longitudinal axis, a rib of the ribs defining three or more guiding apertures extending axially through the rib and being circumferentially distributed around the longitudinal axis, the ribs being interconnected to one another;
cables extending through the guiding apertures, ends of the cables attached to the central conduit proximate the nozzle, the cables configured to be engaged to an actuator for controlling motion of the endoscopic printing head.

12. The endoscopic printing head of claim 11, wherein the ribs are interconnected to one another by longitudinal members extending axially relative to the longitudinal axis from one of the ribs to an adjacent one of the ribs.

13. The endoscopic printing head of claim 12, wherein the longitudinal members include a pair of longitudinal members between a first rib and a second rib of the ribs, longitudinal members of the pair being circumferentially offset from one another by about 120 degrees, the pair of the longitudinal members being clocked relative to a second pair of the longitudinal members disposed between the second rib and a third rib.

14. The endoscopic printing head of claim 11, wherein the ribs define central passages sized to receive the central conduit, the guiding apertures opening to the central passages.

15. The endoscopic printing head of claim 14, wherein the central conduit is a tube received through the central passages of the ribs, the cables being isolated from one another by a cooperation of the ribs and the tube.

16. The endoscopic printing head of claim 11, wherein the ribs are interconnected to one another by an outer shell extending around and secured to peripheries of the ribs.

17. The endoscopic printing head of claim 16, wherein the outer shell is made of an elastic material.

18. The endoscopic printing head of claim 16, wherein spaces defined between the outer shell, the central conduit, and the ribs are filled with an elastic material.

19. The endoscopic printing head of claim 16, wherein the central conduit is separated from the guiding apertures.

20. The endoscopic printing head of claim 11, wherein the ends of the cables are secured to the nozzle, the ends of the cables secured to the central conduit via the nozzle.
